(19) United States
(12) Patent Application Publication
(10) Pub. No.: US 2023/0364286 A1
(43) Pub. Date: Nov. 16, 2023

(54) STERILIZATION METHOD

(71) Applicant: Noxilizer, Inc., Hanover, MD (US)

(72) Inventor: David Opie, Hanover, MD (US)

(21) Appl. No.: 18/316,681

(22) Filed: May 12, 2023

Related U.S. Application Data
(60) Provisional application No. 63/341,912, filed on May 13, 2022.

(51) Int. Cl. A61L 2/20; A61L 2/24

(52) U.S. Cl. CPC A61L 2/20; A61L 2/24; A61L 2101/20

(57) ABSTRACT

Methods and systems for sterilizing medical devices including, but not limited to, a pre-filled syringe. A method for sterilizing a pre-filled syringe may comprise placing the pre-filled syringe in a sterilization chamber and performing a plurality of pulsed sterilization steps. Each pulsed sterilization step may comprise drying the sterilization chamber, humidifying the sterilization chamber, evacuating the sterilization chamber to a target pressure, introducing a quantity of NO2 to the sterilization chamber from a buffer tank that is selectively fluidly linked to the sterilization chamber, passing a predetermined quantity of air through the buffer tank and into the sterilization chamber to aid in rinsing NO2 from the buffer tank into the sterilization chamber, and after the quantity of air is passed through the buffer tank, holding the sterilization chamber for a dwell period at a dwell pressure.

### CROSS REFERENCE TO RELATED APPLICATIONS

The present application claims the benefit of and priority to U.S. Prov. Pat. App. No. 63/341,912, filed May 13, 2022, titled STERILIZATION METHOD, the disclosure of which is incorporated herein by reference.

### TECHNOLOGY FIELD

The present application relates generally to a sterilization method, and in particular, to a sterilization method using NO2 gas.

### BACKGROUND

A wide variety of medical devices have been developed for medical use. Some devices are fully implantable (for example, orthopedic implants, stents and various electrical stimulators) to replace or repair bone, in the bloodstream or elsewhere in the body. Other devices are introduced and removed from the body in a single procedure (for example, endoscopes, catheters, and guidewires). Still other devices are used for introducing materials to or extracting material from the body (for example, syringes). Still other products are used in repairing or otherwise treating the body (for example, sutures and various staples).

Conventionally, high-pressure steam sterilization (hereinafter, simply referred to as “AC”) and ethylene oxide gas sterilization (hereinafter, simply referred to as “ETO sterilization”) have been widely used as sterilizing methods of medical instruments. Gamma radiation may also be used for a range of devices.

AC is a sterilization method in which an item to be sterilized is exposed under a high temperature at approximately 121 to 135° C. and has been widely used for medical instruments made of materials such as metal and glass. However, there is a disadvantage that limitations exist in items to be sterilized since sterilization is performed under a high temperature condition. For example, heat labile materials such as some plastics cannot be sterilized by AC. Some products may contain heat sensitive materials or therapeutic molecules and/or cells, such as pre-filled syringes, and may not be suitable for AC sterilization.

ETO sterilization can be used for plastics since it can be performed at a lower temperature of 70° C. or below. However, due to its toxicity at low concentration and risk of explosion, there is a disadvantage that ETO needs to be securely stored so as not to cause a problem associated with hygienics and safety, and sufficient care needs to be taken in handling. There are hazards associated with ETO off-gassing from ETO sterilized products as well. In addition, when ETO is supplied from a tank (cylinder) to a sterilizing apparatus via a pipe, the occurrence of weight reduction needs to be constantly monitored by measuring the weight of the cylinder for the purpose of preventing unexpected leakage from such as the pipes. Unfortunately, ETO exposure has been linked to alleged cancer risks both within and in the vicinity of sterilization facilities.

Besides those sterilization methods, a sterilization method using hydrogen peroxide has been used. Hydrogen peroxide is simple to use and manage as compared with ETO and is useful from the safety perspective. However, since hydrogen peroxide is used in the form of hydrogen peroxide vapor at or near the saturation partial pressure for hydrogen peroxide, scaling this method of sterilization is challenging due to the need to compensate for inhomogeneous vapor distribution throughout the sterilization chamber.

What may be desirable is a sterilization method that permeates detail portions of a device and can be performed at lower temperatures with reduced risk of toxicity and explosion.

### SUMMARY

This disclosure provides methods of sterilizing devices, such as, but not limited to medical devices. This summary is not intended to describe each disclosed embodiment or every implementation of the invention.

A first illustrative and non-limiting example takes the form of a method of sterilizing a pre-filled syringe, comprising: placing the pre-filled syringe in a sterilization chamber; and performing a plurality of pulsed sterilization steps, wherein each pulsed sterilization step comprises: drying the sterilization chamber with the pre-filled syringe therein; after drying the sterilization chamber to a target, humidifying the sterilization chamber; after humidifying the sterilization chamber, evacuating the sterilization chamber to a target pressure; introducing a quantity of NO2 to the sterilization chamber from a buffer tank that is selectively fluidly linked to the sterilization chamber; passing a predetermined quantity of air through the buffer tank and into the sterilization chamber to aid in rinsing NO2 from the buffer tank into the sterilization chamber; and after the quantity of air is passed through the buffer tank, holding the sterilization chamber for a dwell period at a dwell pressure.

Additionally or alternatively, the dwell pressure exceeds the target pressure by at least 150 Torr. Additionally or alternatively, the target pressure is in the range of about 200 to about 500 Torr, and the dwell pressure is about 600 Torr. Additionally or alternatively, the sterilization chamber has a thermal capacity sufficient to prevent a change in temperature of the pre-filled syringe during the sterilization process to less than 5 degrees C. Additionally or alternatively, the pre-filled syringe has contents the temperature of which does not vary by more than 3 degrees C. during the sterilization process. Additionally or alternatively, the pre-filled syringe contents are maintained in a temperature range of 2-15 degrees C. Additionally or alternatively, a concentration of the NO2 when stored in the buffer chamber is about 100 times a concentration of NO2 after introduction into the sterilization chamber. Additionally or alternatively, a resultant concentration of NO2 in the sterilization chamber during the dwell step is in the range of 2-20 mg/L. Additionally or alternatively, the predetermined quantity of air is in the range of 4-8 times a volume of the buffer tank. Additionally or alternatively, the predetermined quantity of air is dry air. Additionally or alternatively, the method may include rinsing the sterilization chamber with air following the dwell step while monitoring a residual gas in the chamber using a residual gas sensor until a residual gas concentration falls below a predetermined safety threshold.

Additionally or alternatively, a circulation means is provided for recirculating air in the chamber, and the steps of humidifying and introducing the quantity of NO2 are performed by mixing with the recirculating air while the chamber is at a pressure below ambient pressure. Additionally or alternatively, the introduced NO2 is at least partly converted to other chemical products during sterilization, the other chemical products including at least HONO, and the method further comprises monitoring a concentration of HONO during the sterilization procedure, comparing the concentration of HONO to one or more thresholds, and determining that the sterilization procedure is incomplete if the concentration of HONO does not meet the one or more thresholds. Additionally or alternatively, the step of introducing the quantity of NO2 to the sterilization chamber from the buffer tank is performed by: determining first pressure in the sterilization chamber; monitoring a second pressure in the buffer tank; adding air to the buffer tank until the second pressure exceeds the first pressure; and opening a valve between the buffer tank and the sterilization chamber.

Another illustrative and non-limiting example takes the form of a method of sterilizing a pre-filled syringe, comprising: placing the pre-filled syringe in a sterilization chamber; and performing a plurality of pulsed sterilization steps, wherein each pulsed sterilization step comprises: drying the sterilization chamber with the pre-filled syringe therein; after drying the sterilization chamber to a target, humidifying the sterilization chamber to a target humidity level; after humidifying the sterilization chamber, evacuating the sterilization chamber to a target pressure in the range of about 200 to about 500 Torr; introducing a quantity of NO2 to the sterilization chamber from a buffer tank that is selectively fluidly linked to the sterilization chamber; passing a predetermined quantity of air through the buffer tank and into the sterilization chamber to aid in rinsing NO2 from the buffer tank into the sterilization chamber; and after the quantity of air is passed through the buffer tank, holding the sterilization chamber for a dwell period at a dwell pressure of about 600 Torr or more; wherein a concentration of the NO2 in the sterilization chamber during the dwell step is in the range of about 2-20 mg/L.

Additionally or alternatively, the dwell pressure exceeds the target pressure by at least 150 Torr. Additionally or alternatively, a concentration of the NO2 when stored in the buffer chamber is about 100 times a concentration of NO2 after introduction into the sterilization chamber. Additionally or alternatively, the predetermined quantity of air is in the range of 4-8 times a volume of the buffer tank. Additionally or alternatively, the step of introducing the quantity of NO2 to the sterilization chamber from the buffer tank is performed by: determining first pressure in the sterilization chamber; monitoring a second pressure in the buffer tank; adding air to the buffer tank until the second pressure exceeds the first pressure; and opening a valve between the buffer tank and the sterilization chamber.

Another illustrative and non-limiting example takes the form of a method of sterilizing an object, comprising: placing the object in a sterilization chamber; and performing a plurality of pulsed sterilization steps, wherein each pulsed sterilization step comprises: drying the sterilization chamber with the pre-filled syringe therein; after drying the sterilization chamber to a target, humidifying the sterilization chamber; after humidifying the sterilization chamber, evacuating the sterilization chamber to a target pressure; introducing a quantity of NO2 to the sterilization chamber from a buffer tank that is selectively fluidly linked to the sterilization chamber; passing a predetermined quantity of air in the range of 4-8 times a volume of the buffer tank through the buffer tank and into the sterilization chamber to aid in rinsing NO2 from the buffer tank into the sterilization chamber; and after the quantity of air is passed through the buffer tank, holding the sterilization chamber for a dwell period at a dwell pressure; wherein a concentration of the NO2 in the sterilization chamber during the dwell step is in the range of about 2-20 mg/L.

Additionally or alternatively, the step of introducing the quantity of NO2 to the sterilization chamber from the buffer tank is performed by: determining first pressure in the sterilization chamber; monitoring a second pressure in the buffer tank; adding air to the buffer tank until the second pressure exceeds the first pressure; and opening a valve between the buffer tank and the sterilization chamber. Additionally or alternatively, the dwell pressure exceeds the target pressure by at least 150 Torr. Additionally or alternatively, the object is a medical device. Additionally or alternatively, the medical device is disposed within a packaging.

Another illustrative and non-limiting example takes the form of a method of performing sterilization using NO2, comprising: preparing a sterilization chamber containing a product to be sterilized by placing the sterilization chamber in a known state; introducing a quantity of NO2 to the sterilization chamber along with a quantity of humidity, wherein the NO2 and humidity interact in the sterilization chamber to product chemical products of the sterilization process; after introducing the quantity of NO2 and humidity to the chamber, but before evacuating the chamber, using a chemical sensor to monitor a concentration of at least one of the chemical products of the sterilization process during a dwell step; comparing the monitored concentration to a process target; determining whether the dwell step is or has achieved a sterilization goal; and if the dwell step is or has not achieved the sterilization goal, generating an alert to an operator or performing a corrective action; if the dwell step is or has achieved the sterilization goal, recording in memory an indication of success of the dwell step.

Additionally or alternatively, the step of using a chemical sensor to monitor a concentration of at least one of the chemical products includes sensing a concentration of HONO in the sterilization chamber. Additionally or alternatively, the step of comparing the monitored concentration to a process target is performed by comparing the monitored concentration over time to a model of the modeled concentration developed during a verification/validation process, wherein the model is stored in a memory of a controller for the sterilization chamber. Additionally or alternatively, the step of performing a corrective action includes modifying the state of the chamber during the dwell step. Additionally or alternatively, the step of performing a corrective action includes storing an indication that the dwell step has failed and repeating the preparing, introducing and dwell steps. Additionally or alternatively, the step of performing a corrective action includes adjusting a parameter to be used in a subsequent iteration of the preparing, introducing and dwell steps. Additionally or alternatively, the step of performing a corrective action includes extending a duration of the dwell step.

### BRIEF DESCRIPTION OF THE DRAWINGS

The invention may be more completely understood in consideration of the following detailed description of various embodiments in connection with the accompanying drawings, in which:

FIG. 1 is schematic perspective view of an illustrative sterilization chamber;

FIG. 2 is a block diagram of an illustrative sterilization system and gas supply system;

FIG. 3 is a block diagram of an illustrative method of sterilizing an object; and

FIG. 4 is a block diagram for an illustrative dwell period monitoring and correction procedure.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure. The recitation of numerical ranges by endpoints includes all numbers within that range (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Nitrogen dioxide (hereinafter, NO2) has been found to exhibit a beneficial sterilizing effect. An object of the present disclosure is to provide a sterilization method and system which may be used for sterilizing items, such as, but not limited to, scissors, forceps, needles, cannulas, scalpels, tubing, drug delivery devices, filled syringes, empty syringes, staples, implantable medical devices (anchors, embolism coils, stents, catheters, ports, leads, implantation tools, stimulators, pumps, ventricular assist devices, etc.), as well as other medical devices, etc. Endoscopes and other visualization systems, balloon catheters, guide catheters, electro-therapy devices, stylets, implantation tools, filters, baskets and other medical devices may also be sterilized by the methods discussed herein.

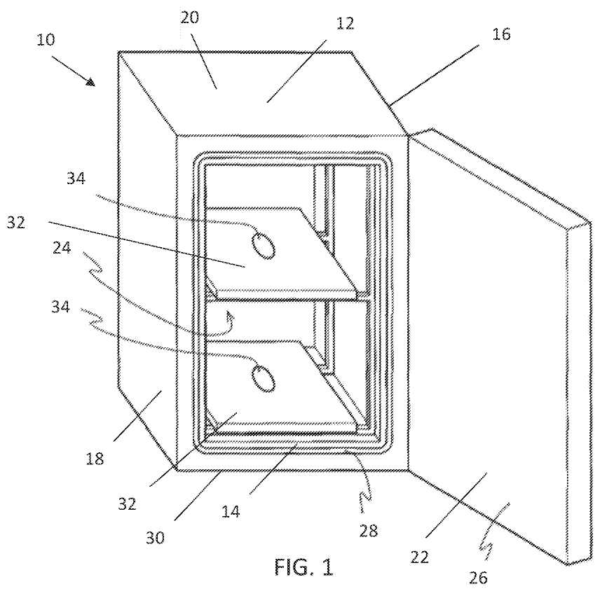

It is contemplated that an item to be sterilized may be placed inside of a sterilization chamber. FIG. 1 is a schematic view of an illustrative sterilizing chamber 10. In the illustrated embodiment, the sterilizing chamber 10 includes a top wall 12, a bottom wall 14 opposite the top wall 12, a first lateral side 16, a second lateral side 18 opposite the first lateral side 16, a back side 20, and a front side 22 opposite the back side 20 (in a closed configuration). The sterilizing chamber 10 may be formed, at least in part, from a material which is not likely to be corroded and/or degraded by the NO2 sterilizing environment. For example, the sterilizing chamber 10 may be formed from stainless steel, a nickel-chrome alloy, an unsaturated polyester resin, etc. In some cases, different materials may be used for different parts of the sterilizing chamber 10. The same or similar materials may be used as well in the pre-chamber and/or buffer tank further discussed below. While the sterilizing chamber 10 has been described and illustrated as having a generally rectangular prismatic shape, the sterilizing chamber 10 may take any shape desired, such as, but not limited to spherical or cylindrical. While not explicitly shown, the sterilizing chamber 10 may be secured or coupled to a base such that the sterilizing chamber 10 is stably supported.

The front side 22 may include or be formed from a movable door 26 configured to open and close to allow access to an interior sterilization cavity 24 of the sterilizing chamber 10 through an access opening 30. The interior sterilization cavity 24 may be defined by an inner surface of each of the walls 12, 14, 16, 18, 20, 22. The door 26 and/or sterilizing chamber 10 may include a sealing material 28 disposed about a periphery of the access opening 30. The sealing material 28 may provide a gas-tight seal when the door 26 is in a closed configuration (not explicitly shown). In some embodiments, the sealing material 28 may be selected for corrosion resistance and pressure tightness. In an illustrative embodiment, a fluorine containing elastomer may be used for the sealing material 28. While not explicitly shown, the door 26 may include a locking mechanism or an interlock which prevents the door 26 from being opened under certain conditions. For example, if a concentration of NO2 gas is equal to or greater than a predetermined level (e.g., a level that may be harmful to people and/or in excess of a regulated limit), the interlock may prevent the door 26 from being opened. In some cases, the interlock may be in electronic communication with a NO2 sensor positioned within the sterilization cavity 24.

The interior sterilization cavity 24 may include one or more shelves 32 for placing an item 34 to be sterilized on. While the sterilizing chamber 10 is illustrated as including two shelves 32, the sterilizing chamber 10 may include more than two or fewer than two shelves as desired. It is further contemplated that other mechanisms for receiving the items 34 to be sterilized may be used as desired. In one example, hooks may be used to hang items from. In another example, vertical racks or slots may be provided to receive devices to be held in a vertical position. Racks, shelves, hooks or other receiving structures may be adapted to receive medical devices in an unpackaged or packaged form. For example, sterilization may be performed with a device held in a tray, with or without a gas permeable cover. In another example, the devices may be packaged for sterilization and loaded in cartons and boxes that are compatible with the NO2 process. These cartons and/or boxes may be transported on pallets and placed in the sterilization chamber 10 on the pallets.

It is further contemplated that the interior sterilization cavity 24 may have volume in the range of about 20 liters (L) to 5000 L, though smaller or larger installations are also possible. The sterilizing chamber 10 may be portable in some examples, and may have wheels for example to allow it to be moved, and may have sterilization cavity volumes of under 100 L or smaller. That is, sterilization may be performed with industrial installations that take up large rooms, as well as in smaller, self-contained and/or portable units. In some examples, a portable chamber may have a volume in the range of about 20 to 25 L. Ports (not shown) may be provided for air-tight coupling to tubes, hoses, etc. that allow the introduction of gasses/fluids into the sterilization chamber as well as removal of gasses/fluids, as desired. Chemical, pressure, temperature, humidity and/or other sensors maybe provided in or on the interior walls of the sterilization chamber 10, and electrical or other connections to such sensors may be routed along and/or through the walls and/or door of the sterilization chamber, as desired. For example, humidity sensors, where the sensing element is in the chamber or in fluid communication with the chamber interior may be based on any known sensing technology, including but not limited to optical, resistive, capacitive, acoustic, resonant, etc. Sensors may be provided in the sterilization chamber at one or more locations for sensing one or more of the chemical products of the sterilization process, for example, NO, HONO, N2O3, and/or NO2 may be monitored.

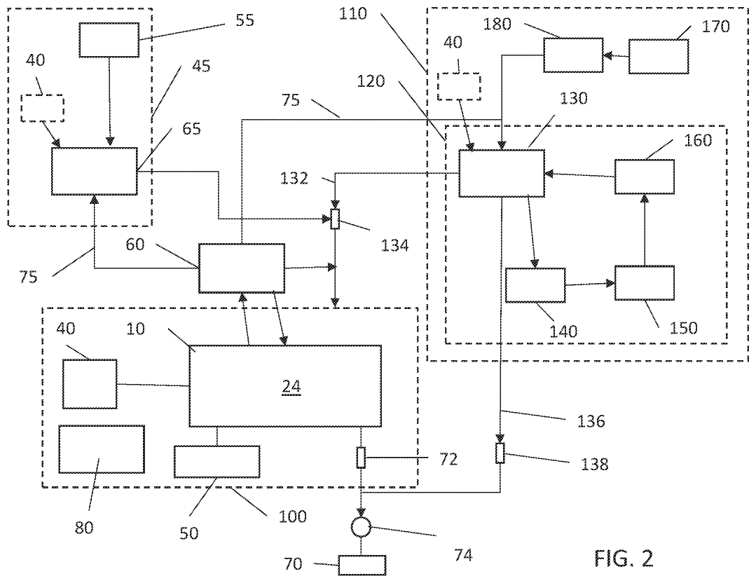

FIG. 2 is a schematic block diagram of an illustrative sterilizing system 100, a first illustrative gas supply system 110, and a second illustrative gas supply system 45. In addition to the sterilizing chamber 10, the sterilizing chamber 10 may include a humidifying apparatus 40 for controlling humidity within the sterilization cavity 24 of the sterilizing chamber 10, a temperature controlling apparatus 50 for controlling a temperature within the sterilization cavity 24 of the sterilizing chamber 10, and a circulation means 60 for dispersing a gas for obtaining a uniform chemical and temperature distribution within the sterilization cavity 24 of the sterilizing chamber 10. The circulation means may include, as desired, a fan/blower coupled to a first pipe or conduit for drawing gas/air from the sterilization cavity 24 which provides an output through a second pipe back to the sterilization cavity 24, where the second pipe may open into the sterilization cavity 24 directly or through a plenum separated from the rest of the sterilization cavity 24 by a screen, grating or diffuser, for example. The circulation means may include as part of a recirculation loop, a scrubber, if desired, which can be used to optionally remove sterilant in the event that sterilant gas concentration in the sterilizing chamber 10 is above a target or setpoint. The sterilizing chamber 10 may be fluidly coupled to the gas supply system 110 for supplying high concentration NO2 to the sterilization cavity 24 of the sterilizing chamber 10. Further, the sterilizing system 100 may include an exhausting apparatus 70 fluidly coupled to the sterilizing chamber 10 for applying a vacuum or suction pressure to the sterilization cavity 24 of the sterilizing chamber 10.

The exhausting apparatus 70 may be operably coupled to the sterilizing chamber 10 via a control valve 72 and a pump 74. Generally, the exhausting apparatus 70 may include a blower, a scrubber, and one or more detectors. In an example, the exhaust may be pulled with a blower into a drum carrying reactive materials that will remove NO2 and other pollutants before exhaust to the atmosphere. A detector may be placed to detect NO2 and/or other process residuals. The one or more detectors may detect NO2 or other gases at the exhaust to determine if the gas is safe to exhaust to atmosphere. When it is desired to apply a vacuum to the sterilization cavity 24 of the sterilizing chamber 10 either to lower a pressure within the sterilization cavity 24 or to evacuate the sterilization cavity 24 after a sterilization procedure, the control valve 72 may be opened to fluidly couple the sterilization cavity 24 and the pump 74. The pump 74 may then be activated to draw gas from the sterilization cavity 24 to the exhausting apparatus 70. It is contemplated that when NO2 gas is exhausted from the sterilization cavity 24 following a sterilization procedure, the exhaust gas may be treated prior to exhausting to the ambient environment. For example, the exhaust gas may be treated with an ozonizer and a nitric acid filter that are downstream of the exhausting apparatus 70. The ozonizer may create ozone which reacts with NO2 to generate dinitrogen pentoxide (N₂O₅). Subsequently, the dinitrogen pentoxide and nitric acid (generated in the sterilization cavity 24) are absorbed by the nitric acid filter. In some cases, the nitric acid filter may include sodium permanganate (NaMnO4) and a layer of activated charcoal. The activated charcoal may not react with the nitric acid, but rather may adsorb the nitric acid and release the nitric acid at a slower rate which lowers the concentration of nitric acid to a safe level. Other approaches may be taken to scrubbing or otherwise removing NO2 from exhaust gasses. For example, the exhaust may be bubbled through water to form nitric acid (HNO₃). The nitric acid may then be neutralized. In yet another example, molecular sieves may be used to capture and reuse the NO2 gas.

The humidifying apparatus 40 may be operably coupled to the sterilization cavity 24 of the sterilizing chamber 10 and/or optionally, to a pre-chamber (or buffer tank) 65 or buffer tank 130 fluidly coupled to the sterilization cavity 24. Humidifying apparatus 40 being coupled to the prechamber 65 or buffer tank 130 may be omitted in some examples. In some cases, a recirculation loop 75 may allow water vapor to be added slowly to the sterilization cavity 24 to avoid localized concentrations of water vapor. For example, an evaporator of the humidifying apparatus 40 may be fluidly coupled to the sterilization cavity 24 of the sterilizing chamber 10, the pre-chamber 65, and/or the buffer tank 130. The humidifying apparatus may use a steam source rather than an evaporator, if desired. The circulation means 60 may pull air, water vapor, etc. out of the sterilization cavity to the pre-chamber 65 or buffer tank 130 and push additional air, water vapor, etc. back into the sterilization cavity 24. The recirculation loop 75 is active to mix the introduced humidity with the dry air and avoid local high concentrations. It is contemplated that the NO2 source will determine which of the pre-chamber 65 or buffer tank 130 is used. For example, if NO2 is provided in a canister 55, the pre-chamber 65 may be used. If NO2 is generated using the gas supply system 110, the buffer tank 130 may be used. In some examples, only one of the first and second gas supply systems 45, 110 are provided to a given system. Both are shown in the illustrative example to allow for explanation of more than one way of generating and introducing NO2 gas to the sterilization chamber 10.

In some illustrative examples, the process gasses are added using the recirculation loop, mixing, for example, the air recirculated from the sterilization chamber with added dry air, humidified air, NO2, or other process gasses before return to the sterilization chamber. Such addition of gasses to the chamber via the recirculation loop may be done with below-ambient pressure processes. A blower capable of providing recirculation, particularly in the sanitary environment can be specially designed for so doing.

The evaporator may include a stainless-steel pipe around which an electric heater is wrapped, with an insulating material covering the heater and pipe. Water may be placed in the evaporator and heated to in the range of about 50° C. to about 80° C. with the electric heater to generate a vapor. In some cases, an ultrasonic humidifier may be used. The vapor may then be introduced to the sterilization cavity 24 under a decreased pressure (e.g., less than 500 millibar (mbar) or 0.5 atm) to humidify the sterilization cavity 24. It is contemplated that vapor may be introduced to a sterilization cavity 24 that is under an absolute dry state created by applying a vacuum to the sterilization cavity 24 prior to introducing the water vapor. It is contemplated that drying the sterilization cavity 24 will result in less residual water which in turn may allow for a better prediction/estimation of how much water has been added to the sterilization chamber 24. For example, by introducing water into a chamber that is pre-dried thoroughly, process control is increased. Starting from a dry state may allow the ideal gas law (PV=nRT) relationship in combination with pressure and temperature sensors to calculate accurate measures of the introduced molar quantity. The water added may be measured as grams of water per volume or as a relative humidity at a particular temperature (e.g., g/m3, or X % RH@Y° C.). This may create a known state to control the process and prevent/limit condensation. For example, excess steam may penetrate and/or saturate cardboard. Further, placing the sterilization cavity 24 in an absolute dry state may allow an amount of the water vapor introduced into the sterilization cavity 24 to be determined by measuring a value of the pressure increase within the sterilization cavity 24 due to humidification. It is contemplated the pressure within the sterilization cavity 24 may be measured with a pressure sensor (not explicitly shown) positioned within or on a wall of the sterilization cavity 24. Thus, a specific humidity level can be obtained within the sterilization cavity 24 by controlling the heating level of the electric heater and an amount of water in the evaporator. In some cases, the heat capacity (and thus the humidification capacity) of the humidifying apparatus 40 may be increased by filling the stainless-steel pipe with stainless-steel pellets. In some examples, a humidity sensor may be provided.

The temperature controlling apparatus 50 may include a rubber heater that is secured to the walls within the sterilization cavity 24. In other examples, temperature-controlled water may flow through channels or tubes that are in contact with the chamber walls. This may allow for heating and/or cooling of the sterilization cavity 24. The amount of heat generated at the rubber heater and/or via the temperature-controlled water may be controlled to provide a desired temperature within the sterilization cavity 24. For example, a thermocouple disposed within the sterilization cavity 24 or attached to the rubber heater may be operably coupled to the temperature controlling apparatus 50 to provide the temperature controlling apparatus 50 with a current temperature of the sterilization cavity 24. The temperature controlling apparatus 50 may then increase or decrease power to the rubber heater to raise or lower the temperature to a desired setpoint temperature. In some cases, the temperature within the sterilization cavity 24 may be in the range of approximately 10° C. to about 90° C. However, other temperatures can be used, as desired.

The walls of the sterilization chamber 10 may be thermally insulated, if desired, to allow further control over the temperatures therein. Temperatures may also be controlled by controlling pressure within the sterilization chamber; for example, pumping in a mass of air or water vapor will both raise the pressure in the sterilization chamber and the temperature thereof. A heater in or associated with the chamber 10 can be provided as well as a cooling apparatus. Cooling and heating may also be performed using a heat pump. Cooling may be performed using a Peltier thermoelectric cooler, for example, or a refrigerant system, as needed. Where room temperature would suffice, cooling and/or warming can be achieved by circulating air against the outside of the chamber, assuming thermal conductivity of the chamber walls is high. The chamber 10 may be provided with a heat sink apparatus to dissipate temperature changes, such as with one or more blocks of metal in contact with the walls that define the chamber, which may function to quickly dissipate temperature changes. Such a heat sink may be removable.

The circulation means 60 may be configured to circulate the gases/vapors within the sterilization cavity 24 and/or via the recirculation loop 75 to provide uniform gas concentrations and relieve humidity throughout the sterilization cavity 24. Injecting gases, especially gases that might condense, into a flowing stream like that provided by the recirculating loop ensures instant mixing. Water and/or sterilant gases (such as NO2, though others may be used in the apparatus/system as shown) may be injected into the recirculation loop 75 near an outlet of the loop 75 to ensure adequate mixing. Further the external recirculating of gases (e.g., external to the sterilization cavity 24) may be critical for gases that are near saturation or gases that need to be mixed as additional gases are added. For example, the circulation means 60 may reduce variations of the sterilant gas concentration and/or relative humidity that may be caused by temperature differentials within the sterilization cavity 24. In some cases, the gases within the sterilization cavity 24 may be removed and reintroduced back into the sterilization cavity 24 via a bellows pump. However, other circulation means may be used, as desired. For example, a fan may be provided within the sterilization cavity 24 to provide uniform temperature distribution, gas concentrations, and/or relative humidity levels. Alternatively, or additionally, the sterilant gas may be distributed by the convection phenomenon of the sterilant gas heated by the temperature controlling apparatus 50. Further, measuring gases in the recirculating loop 75 may provide good mixing before the gas concentration is measured. By injecting gases nearer to the outflow side (e.g., pre-chamber 65 or buffer tank 130) of this circuit and measuring at the inflow side (e.g., downstream of valve 134, which may be a 2-way or 3-way valve depending on which sterilant gas source(s) are included) real-time gas measurement and control or a close approximation thereof may be utilized. This may be important for the ambient and/or near-ambient pressure cycles, like those used for prefilled syringes. The humidifier may be placed at position 40′, rather than or in addition to other positions 40, to allow infusion at the circulation means 60.

A high concentration dose of NO2 gas may be supplied to the sterilization cavity 24 via the first illustrative gas supply system 110 or the second illustrative gas supply system 45. It is contemplated that one or both of the first or second gas supply systems 110, 45 may be coupled to the sterilization cavity 24. The first gas supply system 110 may generate NO2 while the second gas supply system 45 may utilize a canister or cylinder 55 of NO2.

Generally, the second gas supply system 45 may include a canister or cylinder 55 including liquid NO2 and a pre-chamber 65. The pre-chamber 65 may be used to measure the amount of NO2 gas supplied to the sterilization cavity 24. For example, the pre-chamber 65 may be evacuated and then NO2 added to the pre-chamber 65 from the cylinder 55 using a modulated valve that opens and closes quickly at a low duty cycle (e.g., 10 milliseconds open, 3 seconds closed). The pre-chamber 65 may include one or more of pressure, chemical, temperature, humidity, or other sensors, as desired. Some illustrative systems and methods for introducing a sterilant to a pre-chamber are described in commonly assigned U.S. Pat. No. 8,703,066 entitled STERILIZATION SYSTEM AND METHOD and U.S. Pat. No. 8,017,074, entitled STERILIZATION SYSTEM AND METHOD, the contents of which are hereby incorporated by reference. The pressure of the pre-chamber 65 may then be measured and the moles of NO2 added can be determined using the ideal gas law which has been corrected to account for NO2 not being an ideal gas. Alternatively, or additionally, a measurement system may be used to determine the NO2 concentration. For example, infrared detectors or visible light detectors can determine NO2 concentrations in real-time. A valve positioned between the cylinder 55 and the pre-chamber 65 may be modulated to prevent the NO2 from boiling. In some cases, there may be two valves between the pre-chamber 65 and the sterilization cavity 24 to control the rate of evacuation of the pre-chamber 65. The concentration of the NO2 in the pre-chamber 65 may be in the range of 50 to 150 times, or about 100 times, the concentration of the NO2 after introduction into the sterilization cavity 24. In some embodiments, the pre-chamber 65 may have a volume that is in the range of about 0.5% to about 2.0% of the size of the sterilization cavity 24. The smaller volume of the pre-chamber 65 may help ensure that when NO2 is added to the sterilization cavity 24, the pressure within the sterilization cavity 24 remains below atmospheric pressure. Thus, if there is a leak in the sterilizing chamber 10, air is moved from outside the sterilization cavity 24 to inside the cavity 24 to ensure hazardous gas does not escape the sterilization cavity 24.

Generally, the first gas supply system 110 may include an NO2 gas generating system 120 including a pre-chamber or buffer tank 130, a flow resistive portion 140, a plasma generator 150, and a circulating apparatus 160. The flow resistive portion 140 may be fluidly coupled (e.g., via a pipe) to the downstream side of the buffer tank 130, the plasma generator 150 may be fluidly coupled to the downstream side of the flow resistive portion 140, the circulating apparatus 160 may be fluidly coupled to the downstream side of the plasma generator 150, and the buffer tank 130 may be fluidly coupled to the downstream side of the circulating apparatus 160 to create a continuous circulating path.

A gas mixture including nitrogen and oxygen may be introduced to the NO2 gas generating system 120 through an air inlet portion 170. The nitrogen and oxygen may be dried utilizing a gas drying means 180 before being introduced to the buffer tank 130. The buffer tank 130 may include one or more of pressure, chemical, temperature, humidity, or other sensors, as desired. The circulating apparatus 160 may be operated to circulate a mixture of nitrogen and oxygen through the buffer tank 130, flow resistive portion 140, plasma generating portion 150, and circulating apparatus 160 to generate NO2. In some embodiments, the plasma generating portion 150 may be replaced by a cylinder of liquid NO2. Alternatively, or additionally, NO may be introduced to the NO2 gas generating system. Oxidation of NO in the buffer tank 130 (or other chambers along the loop) then creates NO2, as outlined below.

An intense electric field is formed in a plasma generating portion of the plasma generator 150. Nitrogen and oxygen of the gas mixture experience dielectric breakdown by being excited through the intense electric field (e.g., direct current to microwave frequencies), and are displaced from the molecular state to the low-temperature (non-equilibrium plasma) state. The gas under the low-temperature state has a high reactivity with respect to other gases under the low-temperature state or molecular state. Therefore, when the gas mixture including primarily nitrogen and oxygen is introduced to the plasma generating portion 150, a portion thereof is converted to nitrogen oxides, such as nitrogen monoxide (Equation 1) and nitrogen dioxide (Equation 2), or to ozone (Equation 3). Since the pressure of the circulating gas mixture (NOx gas mixture) decreases when it passes through the flow resistive portion 140, the gas mixture can be displaced to the low-temperature plasma state more stably in the plasma generator 150.

N₂+O₂→2NO Equation 1

N₂+2O₂→2NO₂ Equation 2

3O₂→2O₃ Equation 3

It is noted that the conversion ratio is the largest in the case of Equation 1. A portion of NO generated according to Equation 1 binds with oxygen under the low-temperature plasma state in the plasma generating portion and is converted to NO2, as shown in Equation 4.

2NO+O₂→2NO₂ Equation 4

The NOx gas mixture including NO2 thus generated circulates through the NO2 gas generating system 120 by applying pressure thereon with the circulating apparatus 160 or is retained in the buffer tank 130. During this time, NO generated according to equation 1 reacts stepwise with oxygen in the NOx gas mixture or with the ozone generated according to Equation 3, and is further converted to NO2 as shown in Equations 5 and 6. As a result, the NO2 concentration increases as the gas mixture continues to circulate.

2NO+O₂→2NO₂ Equation 5

NO+O₃→NO₂+O₂ Equation 6

Ozone generated according to Equation 3 reacts with nitrogen in the NOx gas mixture to generate NO, as shown in Equation 7.

N₂+2O₃→2NO+2O₂ Equation 7

This NO is also converted to NO2 by the reactions according to Equations 5 and 6.

In this manner, as the dry gas mixture circulates in the NO2 gas generating system 120 by the operation of the circulating apparatus 160, an NOx gas mixture including NO and NO2 is generated by the reaction of nitrogen and oxygen displaced to the low-temperature plasma (non-equilibrium plasma) state when passing through the plasma generator 150. The NO is converted to NO2 by reacting with oxygen in the NOx gas mixture and ozone, and the concentration of NO2 gradually increases. As a result, a high concentration NO2 gas with the NO2 concentration of from 5,000 to 100,000 ppm is generated. A measurement system may be used to determine the NO2 concentration. For example, infrared detectors or visible light detectors can determine NO2 concentrations in real-time.

The buffer tank 130 may be used to temporarily store the generated high concentration NO2. The buffer tank 130 may be connected to the sterilization cavity 24 of the sterilizing chamber 10 via a gas supply line 132. A control valve 134 in fluid communication with the gas supply line 132 may be selectively opened to allow gas to flow from the buffer tank 130 to the sterilization cavity 24 of the sterilizing chamber 10. To aid in mixing and introduction of the high concentration NO2, an additional quantity of dry air may be introduced to the buffer tank 130 to flush the high concentration NO2 into the sterilization chamber. In some embodiments, the buffer tank 130 may have a volume that is in the range of about 0.5% to about 2.0% of the size of the sterilization cavity 24. In one example, the chamber may have a volume of about 40 L, and the sterilization cavity 24 has a volume in the range of 2000 to 5000 L. The concentration of the NO2 in the buffer tank 130 may be in the range of 50 to 150 times, or about 100 times, the concentration of the NO2 after introduction into the sterilization cavity 24. The smaller volume of the buffer tank 130 may help ensure that when NO2 is added to the sterilization cavity 24, the pressure within the sterilization cavity 24 remains below atmospheric pressure. Thus, if there is a leak in the sterilizing chamber 10, air is moved from outside the sterilization cavity 24 to inside the cavity 24 to ensure hazardous gas does not escape the sterilization cavity 24.

A rinse step may, for example, introduce an amount of air to the buffer tank 65/130 and through the gas supply line 132 into the sterilization chamber, where the quantity of rinse air is in the range of 1-10 times the volume of the buffer tank (determined relative to the pressure in the sterilization chamber itself). The rinse volume may be, in some examples, about 4-8 times the volume of the buffer tank 65/130, or about 6 times the volume of the buffer tank 65/130, again referenced to the pressure in the sterilization chamber itself. A source of dry air, such as shown at 66, may be coupled to either buffer tank 65/130 for this purpose.

In another example, as preparation for the introduction of the NO2 from the pre-chamber and/or buffer tank 65/130 to the sterilization chamber 10, the buffer tank may be at least partly pressurized by the addition of either fresh air or dry air. In an example, a process control allows the addition of air to the buffer tank 65/130 until the pressure in the buffer tank exceeds that of the sterilization chamber before the control valve 134 is opened to release the mix of NO2 and air from the buffer tank 130 into the sterilization chamber 10. Using this order of steps is optional, and may be useful to prevent backflow of air and humidity from the sterilization chamber into the passageway from the buffer tank to the sterilization chamber. Additional air is then injected to the buffer tank in the rinse step. In the rinse step, air (ambient or pre-dried) may be added until the pressure in the sterilization chamber increases by a predetermined amount, such as 20 Torr. The pressure increase may be used to monitor the quantity of added ambient or dry air. The air may be added in a plurality of rinse cycles to increase the pressure.

The buffer tank 130 may also be coupled to a vacuum pump or to the pump 74 and exhausting apparatus 70 via another gas supply line 136 and control valve 138. When it is desired to empty the buffer tank 130, the control valve 138 may be opened to fluidly couple the buffer tank 130 and the pump 74. The pump 74 may then be activated to draw gas from the buffer tank 130 to the exhausting apparatus 70. It is contemplated that when NO2 gas is exhausted from the buffer tank 130, the exhaust gas may be treated prior to exhausting to the ambient environment. For example, the exhaust gas may be treated with an ozonizer and a nitric acid filter. The ozonizer may create ozone which reacts with NO2 to generate dinitrogen pentoxide (N₂O₅). Subsequently, the dinitrogen pentoxide and nitric acid (if present) are absorbed by the nitric acid filter. As noted previously, other methods of eliminating NO2 from the exhaust gas flow may be used. The target may be to reach an acceptable threshold of NO2 concentration based on applicable regulations, to ensure occupational hazards are minimized. The target may also be selected to limit NO2 introduction into the general environment. A similar process may be used to empty the pre-chamber 65, as desired.

In the example shown, the humidifying apparatus 40, temperature controlling apparatus 50, circulating apparatus 60, gas supply line 132, and control valve 72 each have their own connection to the sterilization chamber 10. In other examples, fewer such connections to the sterilization chamber 10 are provided. For example, items 40 and 50 may be coupled to the circulating apparatus 60, if desired, so that four ports are provided (two for the circulating apparatus 60, and one for gas supply line 132 and control valve 72. In other examples, the sterilization chamber has fewer ports, such as two or even a single port. In some examples, two ports are provided, one for allowing fluids/gasses to be introduced, and another for allowing fluids/gasses to be exhausted, so that steps related to exhausting the chamber can be performed with one port allowing fluid entry and another port for fluid exhaust in a continuous manner. These are just examples; any desired number of ports can be provided.

In some examples, a controller 80 may be provided in the form of a microcontroller, microprocessor, application specific integrated circuit (ASIC), or computer, that has inputs for receiving diagnostic signals from pressure and/or temperature sensors, any flow monitoring apparatuses, chemical and humidity sensors, etc., and which may also control various valves, circulating, heating and/or cooling apparatuses, etc. throughout the system. As a specific example, an NO2 sensor may be provided in the sterilization chamber to monitor actual NO2 concentrations during the sterilization process, and the controller 80 may record the output of the NO2 sensor at various stages, including before dose introduction, as the dose is introduced, during dwell and after exhaust.

Memory associated with the controller 80 may store machine executable instructions for monitoring and controlling the ongoing processes of the sterilizer. In an alternative, one or more communication ports, such as USB, infrared, or other coupling ports, or a wireless communication subsystem (WiFi, Bluetooth, RF, cellular, etc.) are provided to allow the system to be connected to a computer, whether local or remote, as desired. While controller 80 is shown as part of the sterilizing system 100, should be understood that controller 80 may also be coupled to components of the gas supply systems 45, 110 to obtain diagnostic information and/or to control operations of the components thereof.

In an example, the controller 80 may be used to obtain and record process control signals associated with each step of a sterilization method. Such a controller 80 may also store a model against which process control signals can be compared. For example, a system model can be used to predict the response of various sensors to process steps. That is, a humidity sensor would be expected to sense rising humidity as water vapor is introduced into the chamber; failure of the humidity sensor to return signals that match the stored model may indicate a fault, leading to a warning signal being generated by the system. Similarly, modelling may provide an indication of expected NO2 levels in the sterilization chamber at various times in the process, and mismatch may be identified by comparison of sensed values to that of the process model, in addition to comparing to thresholds for performance of the actual process. That is, for example, any leakage of the sterilization chamber may be observed by holding the system at a relatively low pressure for a period of time during a conditioning phase in which the sterilization chamber is dried. Pressure, temperature, and/or NO2 levels can be monitored as sterilant is introduced into the sterilization chamber to check for correct operation of the control valves and free flow in associated gas lines from the buffer tank 130 to the sterilization chamber 10.

A process control model may also be used to manage system processes in a predictive manner. For example, by modelling temperature or pressure changes, the controller 80 may open or close valves or other actuators, turn on or off heating apparatuses, etc., before sensed pressure, temperature, humidity and/or chemical concentrations reach desirable targets to prevent overshoot.

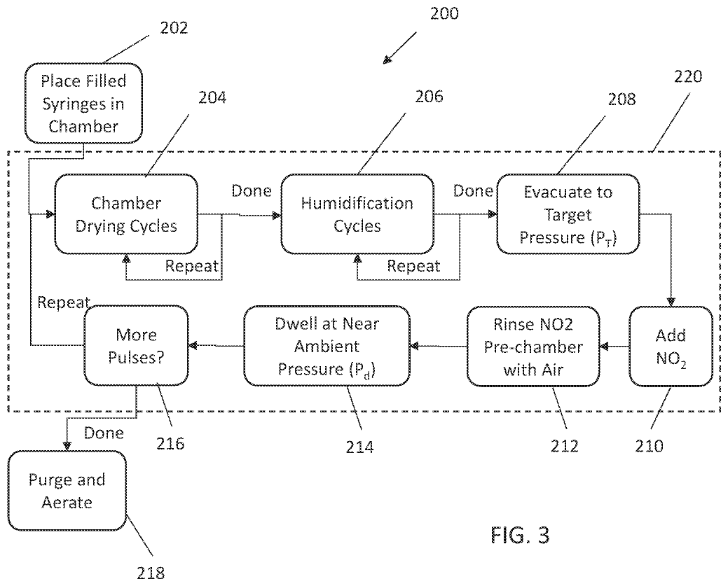

FIG. 3 is an illustrative flow chart of a method 200 for sterilizing an object using the sterilizing system 100. To begin, an object 34 to be sterilized may be placed in the sterilization cavity 24 of the sterilizing chamber 10. In one example, the object 34 may be a filled syringe. The filled syringe may include contents within the barrel thereof. For example, the contents may include a therapeutic agent. The sterilization procedure may be performed such that a temperature of the contents of the syringe does not vary by more than ±5° C. or by more than ±3° C. In other examples, the sterilization process may be performed without concern for temperature changes of the object to be sterilized, or with wider allowed variation, or with an object of actually changing the temperature of the object 34. In some cases, the temperature of the contents may be maintained within a temperature range of about 15-25° C. Once the object has been placed in the sterilization cavity 24, the door 26 of the sterilizing chamber 10 may be closed and locked. The sterilization cavity 24 is then dried using a series of evacuation and fill steps, as shown at block 204. Drying the sterilization cavity 24 may provide a controlled starting place, or controlled process conditions for each “pulse” of the sterilization process, as will be described in more detail herein.

With the sterilization cavity 24 now in a known state, the sterilization cavity 24 is humidified in a controlled manner, with a series of evacuation and fill steps, until a target relative humidity is reached, as shown at block 206. The target relative humidity may be, for example and without limitation, in the range of about 25% to about 90%, or about 40% to about 80%, or about 80%. Target relative humidity may be influenced by process factors including surface temperatures and pressures to be used, where the target relative humidity may be, in some examples, selected to prevent condensation during all phases of the sterilization procedure. In some cases, the relative humidity may be monitored using IR or visible light detectors.

Next, the chamber is depressurized to a target pressure, as shown at block 208. Target pressures PT may be in the range of, for example, 200 Torr to 500 Torr when performing the method with a pre-filled syringe. In some cases, PT may about in the range of about 450 Torr. When humidity is added prior to adding NO2, the humidification may be accomplished by recirculating gas from the sterilization chamber through a humidifying element such as shown at 40′. If preceding steps are performed to leave the dried chamber in a depressurized state, the additional depressurization step at 208 may optionally be omitted.

Next, a quantity of NO2 is introduced into the sterilization cavity 24, as shown at block 210. NO2 may be introduced from one of the gas supply systems 45, 110 with the use of a separate, smaller pre-chamber 65 or buffer tank 130, as described above. In some examples, the sterilization cavity 24 may have a volume in the range of about 20 L to 5000 L, with the corresponding pre-chamber buffer tank 130 having a volume of in the range of about 4 L to 60 L. Other volumes may be used, as desired.

As described above, a concentrated mass of NO2 is placed in the pre-chamber 65 or buffer tank 130 at a lower pressure than the target pressure PT of the sterilization cavity 24, where the lower pressure in the pre-chamber 65 or buffer tank 130 is selected to maintain the NO2 in the buffer tank 130 in its gaseous state and prevent condensation. Next, the pre-chamber 65 or buffer tank 130 may be pressurized by adding ambient or dry air until a pressure in the pre-chamber 65 or buffer tank 130 is reached that is higher than the pressure in the sterilization cavity 24. A valve 134 is then opened to release the NO2 and air mix in the pre-chamber 65 or buffer tank 130 into the sterilization cavity 24. The concentration of the NO2 may be measured using IR detectors or visible light detectors. Additional air is then rinsed through the pre-chamber 65 or buffer tank 130 and into the sterilization cavity 24, as shown at block 212. The volume of added air at the resident pressure may be approximately six times the volume of the pre-chamber 65 or buffer tank 130, thus ensuring mixing of the NO2 and full introduction into the sterilization cavity 24.

Steps 210 and 212 add a quantity of air and NO2 to the sterilization cavity 24, leaving the sterilization cavity 24 with dwell pressure, Pa which may be in the range of about 600 Torr, for example, in the range of 500 Torr to ambient pressure, or even above ambient pressure for example up to about ambient pressure plus 100 Torr. In some examples, the dwell pressure may be in the range of about 550 to about 650 Torr. Dwell pressure may also be understood relative to ambient pressure, being in the range of about 200 Torr below ambient pressure to about 100 Torr above ambient pressure, or in the range of about 100 Torr below ambient pressure to ambient pressure. In some examples, the dwell pressure exceeds the target pressure by at least about 150 Torr. In some examples, the dwell pressure is at ambient pressure, which may be an average ambient pressure for the location, or may be determined by sensing ambient pressure using an external pressure sensor. The quantity of NO2 introduced in step 210 may be sufficient to result in an NO2 concentration in the sterilization cavity 24 in the range of about 2-20 milligrams per liter (mg/L). However, the concentration of NO2 in the buffer tank 130 may be several times higher than the resulting concentration of NO2 in the sterilization cavity 24. For example, assuming the sterilization chamber 10 has a volume of 4000 L, and the buffer tank has a volume of 40 L, the concentration of NO2 in the buffer tank 130 may be in the range of 200 mg/L to attain 2 mg/L concentration in the sterilization cavity 24, or 2000 mg/L to attain 20 mg/L concentration in the sterilization cavity 24. Other values may be used. Specific process parameters may vary depending on the device to be sterilized, including features such as surface contours, materials, material compatibility, whether moving parts are present that may be subject to movement due to pressure changes, the capability of materials, including those of the sterilized object and any material (medicine, biologic, etc.) contained in the sterilized object, to withstand heat, pressure, humidity, and NO2 itself.

The sterilization cavity 24 may be held at stable conditions for a predetermined dwell time, as shown at block 214. For example, the humidity, NO2 concentration, and air in the sterilization cavity 24 may remain fixed for a period of time in the range of about 4 minutes to 15 minutes. However, dwell times less than 4 minutes or greater than 15 minutes may be used, as desired. It should be noted that after the vacuum is applied at step 208, each of steps 210 and 212 raise the pressure in the sterilization cavity 24 from the target pressure PT to the dwell pressure Pd. As a result, the target pressure of step 208 is not the same as the pressure in the sterilization cavity 24 during the dwell step 214.

At the end of any given dwell step, the chamber is evacuated, and NO2 that is evacuated is scrubbed/removed from exhaust gas. The process 220 is repeated for a number of pulses, as indicated at block 216. The number of pulses may range between 2 to 8 pulses. However, in some cases there may be only one pulse or may be more than 8 pulses, as desired. When an additional pulse is called for in the sterilization routine, the process returns to step 204 to again place the sterilization cavity 24 in a known state. When the process returns to block 204, the evacuation and refilling steps that are performed will evacuate NO2 from the prior dwell (214) from the sterilization chamber, which in turn will require treating the exhaust gas that results to reduce the exhausted quantity of NO2 to an environmentally acceptable level.

Once the sterilization routine is complete (e.g., the process 220 has been repeated for a predetermined number of pulses), the sterilization cavity 24 may be purged and aerated, as shown at block 218. The sterilization cavity 24 may be aerated through a series of evacuations or rinsing air through the sterilization cavity 24. For example, a load of air may be pulled through the sterilization cavity 24. To purge the sterilization cavity 24, the gas mixture may be exhausted via the exhausting apparatus 70 to scrub the NO2 and nitric acid before the gas mixture is released to the ambient environment. If desired, the purge and aeration process 218 may include modifying the temperature of the sterilized object such as by using heated or cooled air during purge steps, and by heating or cooling the sterilization chamber walls. The purge and aeration process 218 may comprise rinsing the sterilization chamber with air following the dwell step while monitoring a residual gas in the chamber using a residual gas sensor until a residual gas concentration falls below a predetermined safety threshold. For example, an NO2 sensor in the chamber or in a flow path associated with the chamber, such as in an exhaust flow path, may be used to monitor NO2 levels during aeration until such levels fall below a threshold.

An alternative approach may follow similar steps as shown, but with certain modifications. The sterilization chamber may serve as a decontamination isolator. For such a process, the method shown in FIG. 3 may be followed generally as described above, with some exceptions. The product to decontaminate is added at block 202, and may or may not be pre-filled syringes. One or more drying cycles are performed at 204, which may include following a similar process to that described above. Humidification cycles are then performed at block 206. The evacuation step 208 can be omitted, and NO2 is added at block 210 using a similar process to that described above. To introduce the NO2, slightly pressurized air (10 to 100 Torr above ambient and/or above a sensed or calculated in-chamber pressure) or a blower may be used to drive dry air into the pre-chamber during block 210 and 212. The dwell step 214 can then be performed at ambient pressure. Additional pulses can be performed at block 216 by going back to block 204. Finally, the system is purged at block 218.

In some examples, the success of the process may be monitored based on a concentration of chemical reaction byproduct species. For example, N2O3 is the deadly molecule, which reacts with the DNA/RNA strands and destroys them, after passing through the cell membrane. For example, to form the N2O3, the species that are predicates to that formation are NO and HONO. Concentrations of NO, HONO, N2O3, and/or others may be monitored to determine if N2O3 was present in sufficient concentrations to sterilize the object(s) in the sterilization cavity 24.

In an illustrative example, process monitoring in the sterilization chamber may include monitoring one or more of NO, HONO, N2O3, and/or NO2 in the sterilization chamber during the process as illustrated. In one example, during the dwell step at 214, one or more of the NO, HONO, N2O3, and/or NO2 concentrations in the chamber are monitored with a sensor. For example, NO2 concentration may be monitored against a target concentration in the range of, for example and without limitation, 2-20 mg/L. A particular example monitors for HONO during the dwell step; it is to be anticipated that the concentration of HONO will change during the dwell process as the dwell period advances. In some examples, a model of expected HONO concentration can be developed for a particular sterilization procedure during validation and verification (V/V) of the sterilization procedure. For example, a test run of the sterilization process may be performed and followed by inspection and/or testing of sterilized products to determine whether the sterilization process performed as expected The HONO model developed during such V/V activity may be stored in memory of the system controller 80 (FIG. 2) and used to determine whether a particular run of the process has complied with system monitor requirements. Use of such modeling may allow for elimination, or reduced reliance on, biologic process monitors which can be expensive to create, store, and inspect following use. Alternatively, the concentration of HONO may be compared to a threshold (such as an average during all or a portion of the dwell period, or a peak or minimum concentration, or concentration at the end of the dwell period). Monitoring of NO, N2O3 may be performed instead using a similar approach.

Parameterized process monitoring (the use of measured values rather than or in addition to biologic sample materials), such as monitoring concentrations of one or more of NO2, NO, HONO, and/or N2O3 during sterilization may be used in several ways. For example, when determining whether more pulses are needed at block 216, the controller may determine whether one or more, or a target number, of preceding iterations have met the target concentrations and/or correlated to stored models of concentrations NO, HONO, N2O3, and/or NO2; if not, another pulse may be called for. During the dwell period 214, it may be possible to add one or more materials to the chamber in a small pulse, such as via the pre-chamber/buffer chamber, such as introducing additional NO2 or humidity if a monitored parameter is not matching its target. Likewise, partial venting of the chamber, can be done to reduce chemical or humidity concentrations in the chamber. In another example, additional fresh or dry air may be added to the chamber to reduce or otherwise modify chemical and/or humidity concentration in the chamber more closely match targets. In one example, both venting and addition of dry air may be performed.

In some examples, temperature or pressure in the chamber may be adjusted up or down to affect the rate of chemical reaction in the chamber; typically increasing temperature accelerates the chemical reaction, and cooling the chamber will slow the chemical reaction. Rather than adding or removing material within the chamber, an inflatable bladder in the chamber or on a chamber wall may be inflated or deflated to adjust in-chamber pressure (by reducing or increasing volume in the sealed space) to adjust pressure. The circulation blower may be modulated in response to the tracked chemical or humidity status in the chamber; for example, if the concentration of NO2 remains relatively higher, but the HONO concentration is not tracking to a model, the blower may be activated or blower speed increased to encourage additional mixing in the chamber thereby increasing the rate of chemical reactions generating HONO. These are just examples of steps that can be taken to modulate or correct the HONO concentration, or the concentration of another sterilant and/or chemical product of a sterilization process in the chamber in response to a sensor output indicating that HONO is not matching a modeled parameter trend and/or is not matching a target.

Rather than doing something to adjust concentration of a monitored parameter, the dwell period itself may be adjusted by adding more dwell time is concentrations are below desired targets, or reducing dwell time if concentrations are above target or reach targets earlier than modeled. In some examples, a corrective action may be taken outside of the particular dwell step being monitored, such as by adjusting a concentration, temperature, humidity, or pressure to be used in a subsequent dwell step for the same set of sterilized product, or for use in sterilization of a subsequent product. In still other examples, monitored parameters may be used to trigger an alert to an operator that one or more system components is not working as expected, suggesting for example a clogged valve, inoperative actuator, or other failure or reduced performance.

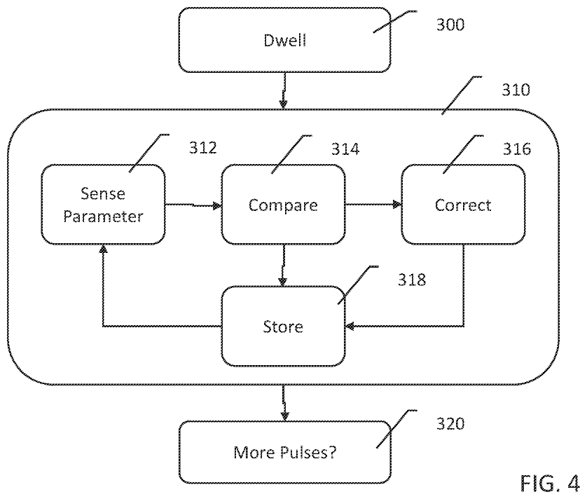

FIG. 4 shows an illustrative example. During the dwell step 300, process parameter monitoring 310 takes place. Parameters (humidity, temperature, concentration of any of NO2, NO, HONO, H2NO3 or other sterilant and/or sterilization process chemical marker) are monitored using sensors at 312, and compared to stored targets and/or models at 314. Comparison may be simple comparison relative to a target, or may include tracking deviation from a trend or target using standard statistics (such as determining if the process stays within a standard deviation of process parameters). If the monitored parameter is out of range, above or below target, tracking or trending away from the model and/or target, a correction may take place at 316. Correction 316 may include a wide range of steps in accordance with the many types of sterilization that can be used as well as the many processes that may result; several corrective actions are described in the preceding discussion. Correction 316 may be omitted in some examples such that steps 312, 314 and 318 are used to monitor the process and generate or trigger one or more alerts regarding operability of the chamber, the state of the sterilization process, or success/failure of a particular iteration/dwell steps in the sterilization process. The monitored data, comparison to targets, trends and/or models, and or any corrective steps taken are then stored to memory at 318 and may become part of the history file for the product(s) being sterilized. Finally, when the dwell period ends, the process optionally moves from block 310 to determine if more pulses are needed at 320. The stored data from block 318 may be used when determining whether or not more pulses are needed.

Several specific examples of products that may be sterilized, and sterilization considerations that apply, follow.

Pre-filled syringes may be sterilized as noted several times above using an NO2 sterilization process. Processes may be performed at temperatures near room temperature (that is, in the range of 10-30 C, or more narrowly, 15-25 C), depending on how susceptible the content of the syringes are to breakdown due to the applicable temperature. Because a syringe typically has a barrel and a plunger associated with the barrel, where the plunger is moveable relative to the barrel, low pressures (such as below 100 Torr, or below 200 Torr, depending on design) may be avoided. In an example the pressure at step 208 may be in the range of 350 to 500 Torr, and a quantity of NO2 and air is added at 210, 212 to achieve an NO2 concentration in the sterilization chamber of 2-40 mg/L, at a pressure of about 550 to 650 Torr.

An implantable electrostimulation device, such as a pacemaker, defibrillator, or neurostimulator, may be sterilized as well. Such systems typically have a port for receiving lead with the port surrounded by plastic material, such as epoxy, with silicone components used to insulate portions of the port, and are often housed in a metallic canister made of, for example, titanium, having a coating thereon such as Titanium Nitride, Iridium Oxide, or any other suitable coating. Other materials may include polysulfone, silicone rubber, and/or silicone medical adhesive and/or biodegradable.

A stent delivery system for delivering cardiac stents which may include an elongated catheter having one or more inflatable balloons at a distal end thereof carrying an expandable metal stent, with the stent being coated with a dissolvable drug coating thereon, may be sterilized. Resorbable stents including poly(L-lactide) (PLLA), poly-D, L-lactide (PDLLA), iron, zinc, magnesium, alloys thereof, and/or others may be sterilized. For lubricious materials, lower humidity levels may be required. In some cases, therapeutic agents may be need to be protected during the sterilization process. For example, an extra layer or coating of the therapeutic agent may be provided on the device.

Other illustrative devices may include orthopedics, medullary nails, pedicle screws, custom implants, 3-D printed implants, metal implants including, but not limited to, nitinol, titanium, chrome, tantalum markers, polymeric implants including, polyethylene (PE), polyetheretherketone (PEEK), polylactic acid (PLA), poly(lactic-co-glycolic acid) (PLGA), poly(caprolactone-co-lactide) (PCLA), or combinations thereof. It is contemplated that the specific water activity level of the nitrate salt of a metal may be used as an indicator of whether a particular metal can be sterilized in an NO2 environment.

Pressures are typically kept below atmospheric pressure during sterilization to avoid release of NO2 in the direct vicinity of the sterilization chamber. In some examples, an NO2 sensor may be provided on the outside of the sterilization chamber as a way of monitoring for escape of NO2, which may impair the actual sterilization process itself as well as creating a potential hazard for workers in the area. As noted above, in some examples, pressures may be at or above ambient levels during a sterilization process; in such cases, an external NO2 sensor may be used, and the space in which the sterilization chamber is provided may be sealed, or, in the alternative, ventilated with the aid of a scrubber to remove NO2 from air exiting the chamber.

Many products may be held in a tray that is enclosed with a gas permeable material, such as spunbound high density polyethylene or polypropylene fibers (Tyvek®, for example), though other materials may be used. Paper can be used, though it is subject to yellowing during the NO2 sterilization process, a result that is generally not desired as sterile products are not expected to have a “weathered” look. However, coated paper, lacquered cardboard, and/or labels may be used without a yellowing effect.

Products requiring refrigeration may be sterilized in the NO2 chamber. In an example, a product maintained at a cool temperature for storage purposes, in the range for example of 1 C to 15 C, can be sterilized using an NO2 sterilization process without requiring that the product be warmed to room temperature. Such pre-warming is needed for other sterilization processes and significantly delays processing time while also exposing product to warm temperatures, accelerating its degradation and/or reducing shelf life.

For NO2 sterilization of cold products, the walls of the sterilization chamber may optionally be cooled by circulating cooled fluid therethrough, as described above. Referring to FIG. 3 again, the product is taken from cold storage and placed in the sterilization chamber 202 and proceeds through the process of drying steps 204 described above. In particular, pressure in the chamber is reduced for example to a pressure in the range of 350 Torr, and dry air is introduced to return the pressure to 500 Torr, all (optionally) with the chamber walls maintained at a cool temperature of, for example, 1 C to 15 C. After a number of drying cycles (anywhere from 1 to 60 drying cycles may be performed), a dose of humidity is introduced into the chamber 206, keeping the relative humidity in the range of about 25% to about 90%, or about 40% to about 80%, for example. To prevent or avoid localized condensation, humidity may be introduced in a series of iterations, such as by removing air to get to a vacuum target (which will also cool the chamber), introducing a dose of water vapor mixed with air (which will warm the chamber temporarily), and repeating the process.

The cooled, humidified chamber then receives the NO2 dose at 210, and the NO2 pre-chamber or buffer tank is rinsed with dry air. These steps will increase pressure in the sterilization chamber and also temporarily raise the temperature in the chamber. The dwell step 214 is then performed, and the process may be repeated until sufficient pulses 216 have been completed, upon which time the chamber is purged and aerated 218. It is contemplated that the process may require a longer dwell period 214, or higher NO2 concentration, than would be needed at higher temperatures, or that more pulses 216 may be needed. Such a process can be performed, for example, with a pre-filled syringe containing a product that requires cold conditions for longevity, effectiveness, etc. Some examples may include various vaccines, biologic products, drugs, etc., including for example certain ophthalmic products. Even if the sterilization chamber process requires more time when performed cold, the overall sterilization processing time can be reduced by avoiding the product pre-warming (which may take up to 72 hours for palletized products) and cooling afterward, and will also avoid product degradation and/or shortening of shelf life associated with the time spent outside of refrigerated storage.

Piping into and out of the chamber may be temperature controlled in some examples. For example, when delivering humidity and or water vapor to the chamber, piping walls may be warmed to prevent condensation thereon within the pipe (where flow may occur at higher pressure than will be encountered in the overall chamber). In some examples, the supply of dry air provided to the chamber may be delivered through cooled pipes or after passing the dry air through a heat exchanger to cool the infused air, aiding in temperature control within the chamber.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A method of sterilizing a pre-filled syringe, comprising:
placing the pre-filled syringe in a sterilization chamber; and
performing a plurality of pulsed sterilization steps, wherein each pulsed sterilization step comprises:
drying the sterilization chamber with the pre-filled syringe therein;
after drying the sterilization chamber to a target, humidifying the sterilization chamber;
after humidifying the sterilization chamber, evacuating the sterilization chamber to a target pressure;
introducing a quantity of NO2 to the sterilization chamber from a buffer tank that is selectively fluidly linked to the sterilization chamber;
passing a predetermined quantity of air through the buffer tank and into the sterilization chamber to aid in rinsing NO2 from the buffer tank into the sterilization chamber; and
after the quantity of air is passed through the buffer tank, holding the sterilization chamber for a dwell period at a dwell pressure.

2. The method of claim 1, wherein the dwell pressure exceeds the target pressure by at least 150 Torr.

3. The method of claim 1, wherein the target pressure is in the range of about 200 to about 500 Torr, and the dwell pressure is about 600 Torr.

4. The method of claim 1, wherein the sterilization chamber has a thermal capacity sufficient to prevent a change in temperature of the pre-filled syringe during the sterilization process to less than 5 degrees C.

5. The method of claim 1, wherein a concentration of the NO2 when stored in the buffer chamber is about 100 times a concentration of NO2 after introduction into the sterilization chamber.

6. The method of claim 1, wherein the predetermined quantity of air is in the range of 4-8 times a volume of the buffer tank.

7. The method of claim 1, wherein a circulation means is provided for recirculating air in the chamber, and wherein the steps of humidifying and introducing the quantity of NO2 are performed by mixing with the recirculating air while the chamber is at a pressure below ambient pressure.

8. The method of claim 1, wherein the introduced NO2 is at least partly converted to other chemical products during sterilization, the other chemical products including at least HONO, and the method further comprises monitoring a concentration of HONO during the sterilization procedure, comparing the concentration of HONO to one or more thresholds, and determining that the sterilization procedure is incomplete if the concentration of HONO does not meet the one or more thresholds.

9. A method of sterilizing a pre-filled syringe, comprising:
placing the pre-filled syringe in a sterilization chamber; and
performing a plurality of pulsed sterilization steps, wherein each pulsed sterilization step comprises:
drying the sterilization chamber with the pre-filled syringe therein;
after drying the sterilization chamber to a target, humidifying the sterilization chamber to a target humidity level;
after humidifying the sterilization chamber, evacuating the sterilization chamber to a target pressure in the range of about 200 to about 500 Torr;
introducing a quantity of NO2 to the sterilization chamber from a buffer tank that is selectively fluidly linked to the sterilization chamber;
passing a predetermined quantity of air through the buffer tank and into the sterilization chamber to aid in rinsing NO2 from the buffer tank into the sterilization chamber; and
after the quantity of air is passed through the buffer tank, holding the sterilization chamber for a dwell period at a dwell pressure of about 600 Torr or more;
wherein a concentration of the NO2 in the sterilization chamber during the dwell step is in the range of about 2-20 mg/L.

10. The method of claim 9, wherein the dwell pressure exceeds the target pressure by at least 150 Torr.

11. The method of claim 9, wherein a concentration of the NO2 when stored in the buffer chamber is about 100 times a concentration of NO2 after introduction into the sterilization chamber.

12. The method of claim 9, wherein the predetermined quantity of air is in the range of 4-8 times a volume of the buffer tank.

13. The method of claim 9, wherein introducing the quantity of NO2 to the sterilization chamber from the buffer tank is performed by:
determining first pressure in the sterilization chamber;
monitoring a second pressure in the buffer tank;
adding air to the buffer tank until the second pressure exceeds the first pressure; and
opening a valve between the buffer tank and the sterilization chamber.

14. A method of performing sterilization using NO2, comprising:
preparing a sterilization chamber containing a product to be sterilized by placing the sterilization chamber in a known state;
introducing a quantity of NO2 to the sterilization chamber along with a quantity of humidity, wherein the NO2 and humidity interact in the sterilization chamber to product chemical products of the sterilization process;
after introducing the quantity of NO2 and humidity to the chamber, but before evacuating the chamber, using a chemical sensor to monitor a concentration of at least one of the chemical products of the sterilization process during a dwell step;
comparing the monitored concentration to a process target;
determining whether the dwell step is or has achieved a sterilization goal; and
if the dwell step is or has not achieved the sterilization goal, generating an alert to an operator or performing a corrective action;
if the dwell step is or has achieved the sterilization goal, recording in memory an indication of success of the dwell step.

15. The method of claim 14, wherein the step of using a chemical sensor to monitor a concentration of at least one of the chemical products includes sensing a concentration of HONO in the sterilization chamber.

16. The method of claim 14 wherein the step of comparing the monitored concentration to a process target is performed by comparing the monitored concentration over time to a model of the modeled concentration developed during a verification/validation process, wherein the model is stored in a memory of a controller for the sterilization chamber.

17. The method of claim 14, wherein the step of performing a corrective action includes modifying the state of the chamber during the dwell step.

18. The method of claim 14, wherein the step of performing a corrective action includes storing an indication that the dwell step has failed and repeating the preparing, introducing and dwell steps.

19. The method of claim 14, wherein the step of performing a corrective action includes adjusting a parameter to be used in a subsequent iteration of the preparing, introducing and dwell steps.

20. The method of claim 14, wherein the step of performing a corrective action includes extending a duration of the dwell step.
